(19) United States
(12) Patent Application Publication
(10) Pub. No.: US 2020/0082922 A1
(43) Pub. Date: Mar. 12, 2020

(54) Method And System For Patient Information Safety

(71) Applicant: Steven V. Bacastow, Cumming, GA (US)

(72) Inventor: Steven V. Bacastow, Cumming, GA (US)

(73) Assignee: QuickVault, Inc., Cumming, GA (US)

(21) Appl. No.: 16/680,648

(22) Filed: Nov. 12, 2019

Related U.S. Application Data
(63) Continuation of application No. 15/072,112, filed on Mar. 16, 2016, now abandoned.
(60) Provisional application No. 62/135,865, filed on Mar. 20, 2015.
(60) Provisional application No. 62/135,857, filed on Mar. 20, 2015.

(51) Int. Cl. G16H 10/60; H04L 29/06

(52) U.S. Cl. CPC G16H 10/60; H04L 63/10

(57) ABSTRACT

The present invention relates to a method and system protecting structured and unstructured PHI data as it is shared and moved between authorized and unauthorized devices and among authorized and unauthorized users based on entitlement.

### RELATED APPLICATION

The present application is a continuation application of and claims priority to U.S. patent application Ser. No. 15/072,112, titled “Method and System For Patient Information Safety,” and filed Mar. 16, 2016, which claims priority to U.S. Provisional Patent Application No. 62/135,857, titled “Method and System For Patient Information Safety,” filed on Mar. 20, 2015 and U.S. Provisional Patent Application No. 62/135,865, titled “Apparatus, Method and System For HIPAA Wallet,” filed on Mar. 20, 2015. The entire content of the foregoing applications is incorporated herein by reference.

### COPYRIGHT NOTICE

A portion of the disclosure of this patent document may contain material, which is subject to copyright protection. The copyright owner has no objection to the facsimile reproduction by anyone of the patent document or patent disclosure as it appears in the U.S. Patent and Trademark Office patent file or records, but otherwise reserves all copyright rights whatsoever.

### FIELD OF THE INVENTION

The present invention relates to a method and system for protecting patient data as it is shared and moved between authorized and unauthorized devices and among authorized and unauthorized users.

### BACKGROUND OF THE INVENTION

Many medical care providers rely on paper and plastic-based processes for key patient engagement processes. For example, in a typical use case, a patient is required to show proof of insurance upon arrival at a medical care provider such as a hospital or doctor's office. This proof of insurance is generally provided by means of a plastic or paper insurance card that includes the patient's policy number along with a phone number for contacting the insurance provider. Next, a representative of the medical care provider contacts the insurance provider to determine if the patient has coverage in place and to determine the amount due from the patient (the co-payment). Next, the patient is required to authorize the disclosure of medical records to other providers, again generally using a paper form. This authorization is generally a blanket authorization to release medical records of the patient to internal parties, and outside parties such as labs and referred physicians. After the medical procedures are completed, the patient is required to pay the full amount or a co-payment amount using a payment tender method such as cash, check, or plastic credit or debit card.

These legacy paper and card based processes are inefficient and can lead to unauthorized disclosures of patient information including the patient's medical condition, medical treatment, or medical payment. However, notwithstanding the difficulty of doing so with a paper based process, hospitals and other healthcare provider enterprises are required to protect patient data according to strict regulatory requirements such as Health Insurance Portability and Accountability Act (HIPAA). HIPAA for example requires that all patients be able access their own medical records, correct errors or omissions, and be informed about how personal information is shared and used. Other HIPAA provisions involve notification of privacy procedures to the patient. Despite the existence of these regulations, healthcare enterprises struggle to comply with the laws due to antiquated systems and methods and the lack of a comprehensive method and system for HIPAA policy enforcement. In fact, most health care organizations do not know where all patient data resides within their organization.

Patient data may be of a structured or un-structured type. Unstructured data is data that is dictated, keyed as text, or scanned and may be in the form of physician notes and other paper documents. Physical security over this unstructured data varies greatly between locations and healthcare providers. Unstructured data may be photo-copied, emailed, sent by courier, or faxed between healthcare providers without controls or audit trails.

Structured patient data may be comprised and stored in systems such as Electronic Medical Record (EMR) Systems and Electronic Healthcare Record (EHR) Systems. However, there are no standards for how patient data is stored and secured within these EMR and EHR systems. Neither are there standard user entitlements which can serve to grant consistent access to patient data based on a patient's prior approval and a need-to-know based on role and medical procedure. Patient's themselves have little or no access to their own protected health information (“PHI”) data and are generally not aware of the formats or locations where their PHI data exists or is disclosed. Finally, a patient's personally identifiable information (“PII”) data is commonly stored unencrypted in the same system which houses their medical history. The combination of PII data such as name, address, social security number with information that discloses one or more of a medical condition, a medical treatment, or a medical payment is considered electronic-PHI or ePHI. A breach of an EMR or EHR system would then provide easy, single-point-of-access to a patent's ePHI data.

There is a lack of interoperability between various EMR systems. Patients receive care from providers that use a variety of EMR systems. Providers change over time as do their systems. In a real example, a first patient, named here as John Doe, was treated by a first doctor for a frozen shoulder, and in the same year the patient was treated by a second doctor for a colonoscopy, and a third doctor (his primary physician) for his annual physical exam. Mr. Doe had previously been treated by a fourth doctor for his prior annual physical exams before a certain date. Mr. Doe's current primary physician determined that Mr. Doe's PSA level was unusual for his age and referred Mr. Doe to a fifth doctor. The fifth (referred physician of a Urologist specialty) requested a complete history of Mr. Doe's blood work including PSA in order to determine the patient's PSA trend over time. However, Mr. Doe's current primary physician was unable to provide these historical records because they were comprised within the EMR system used by the fourth (the former primary physician). Therefore, Mr. Doe was required to contact the fourth physician to obtain a paper copy of these records. Mr. Doe delivered the paper copy of the blood work including the PSA count to the referred Urologist. The blood work also contained disclosures of results that were not needed by the Urologist such as tests for Hemoglobin, Glucose, Creatinine, and results that included Mr. Doe's cholesterol, triglycerides, HDL and other test results that were not relevant for further diagnosis of Mr. Doe's condition. There was no easy way for either Mr. Doe, or Mr. Doe's current primary physician to provide the PSA test results to the Urologist.

In another example involving Mr. Doe, Mr. Doe was treated for a frozen shoulder by an Orthopedist (the first doctor). Mr. Doe was prescribed prescription Ketorolac® as a pain medication following the procedure. Shortly after the procedure related to Mr. Doe's frozen shoulder, Mr. Doe was scheduled to undergo a routine colonoscopy. During pre-operation registration the day before the scheduled colonoscopy, Mr. Doe disclosed that he was taking the prescription Ketorolac®. The gastroenterologist (the second doctor) postponed the colonoscopy procedure because there was a high risk of potential bleeding if a polyp was to be removed. In this example, the patient was required to remember to disclose the prescription drug. Failure to do so could have resulted in an unwanted medical outcome.

A need exists for a method and system that addresses these shortcomings in the current systems and processes by protecting structured and unstructured PHI data as it is shared and moved between authorized and unauthorized devices and among authorized and unauthorized users based on entitlements and wherein medical records are only released based on a need-to-know and wherein the released medical records contain the optimal information needed for proper medical care.

### SUMMARY OF THE INVENTION

The present invention answers these needs by providing an apparatus and system for granting access to and protecting PHI data as it is shared and moved between authorized and unauthorized devices and among authorized and unauthorized users. According to the present invention PII data is tokenized using strong encryption and stored separately from the associated PHI data which comprises the actual medical records of the patient.

In another embodiment, a user entitlements framework is established whereby a healthcare provider, patient or payer may have access to information on a need-to-know basis.

In another embodiment, a common middleware layer may be implemented to provide universal access to PHI data that is originally stored in a variety of EMR and EHR systems.

In another embodiment, a mobile device is equipped to read a signal from a beacon installed at the medical care provider location. The beacon provides information to the mobile device that identifies the identity of the medical care provider and that causes the mobile device to contact a remote HIPAA Security Server. The remote HIPAA Security Server is operable to identify the patient's unique identity using tokenized patient credentials transmitted from the mobile device; the credentials are transmitted to the server using an application comprised within the mobile device.

In another embodiment, the remote HIPAA Security Server is operable to receive tokenized patient PII information and identity information of the medical care provider from a mobile device. Upon receiving the tokenized patient PII information and the identity of the medical care provider, the remote HIPAA Security Server is operable to communicate directly to a computing device located at the medical care provider where the patient has arrived. Communication to the computing device located at the medical care provider may be in the form of an email, text message, or system interface such as a push notification to a mobile device, a web service, xml, or restful interface. A tablet may be provided to the medical care provider in a secure and controlled manner for the purpose of receiving communications from the remote HIPAA Security Server.

In another embodiment, the remote HIPAA Security Server is operable to receive the insurance policy account information from the mobile device; the insurance information is transmitted to the remote HIPAA Security Server using the application comprised within the mobile device of the patient. The remote HIPAA Security Server can contact the insurance provider to verify coverage on behalf of the patient and communicate verification of coverage to the medical care provider. Communication to the computing device located at the medical care provider may be in the form of an email, text message, or system interface such as a push notification to a mobile device, a web service, xml, or restful interface. A tablet may be provided to the medical care provider to receive such communication in a secure and controlled manner.

In another embodiment, the remote HIPAA Security Server is operable to communicate to the application comprised within the mobile device of the patient, the payment or co-payment amount due from the patient. Using the application user interface on the mobile device, the patient can authorize the payment of the amount due. The patient can select the method of payment from pre-registered payment methods that are displayed from the mobile device user interface. Payment methods may be one of card present, card-not-present payments or alternative payment methods. Card-not-present payment methods may include but are not limited to registered Debit, Credit, DDA, PayPal, Mobile Stored Value, ACH or other alternate payment accounts such as Bitcoin. Card present payment methods may include Debit, Credit and other typical card present payment account types that may already be accepted by the medical care provider.

In another embodiment, the security application comprised within the mobile device of the patient may be directly interfaced with an available NFC payment method that is already installed on the mobile device of the patient. Using this embodiment, the security application comprised within the mobile device of the patient can invoke a payment instruction to an installed payment service such as ApplePay®, AndroidPay®, SamsungPay® or another installed NFC payment application. The selected NFC payment application can transmit the previously stored payment credential of the patient or a tokenized, single-use payment credential to an NFC enabled point-of-sale device which is operable to process a payment for the amount due. Embodiments of the present invention are described below by way of illustration. Other approaches to implementing the present invention and variations of the described embodiments may be constructed by a skilled practitioner and are considered within the scope of the present invention.

### BRIEF DESCRIPTION OF THE DRAWINGS

FIG. 1 is an overview of the environment required to support the primary embodiments of the technical environment for patient information safety.

FIG. 2 is a sequence of the exemplary steps involved in granting access to data stored in an EMR system based on role and entitlement.

FIG. 3 is an overview of the environment required to support the primary embodiments required to support the HIPAA Security Application.

FIG. 4 is a sequence of the exemplary steps involved in using the mobile device in conjunction with patient registration and HIPAA disclosure using the HIPAA Security Server.

FIG. 5 is a more detailed description of the patient information middleware.

FIG. 6 is a more detailed description of the database supporting the functions of the patient information middleware.

FIG. 7 is a summary use case that combines embodiments described herein to achieve the primary objective of protecting patient information.

### DETAILED DESCRIPTION OF THE INVENTION

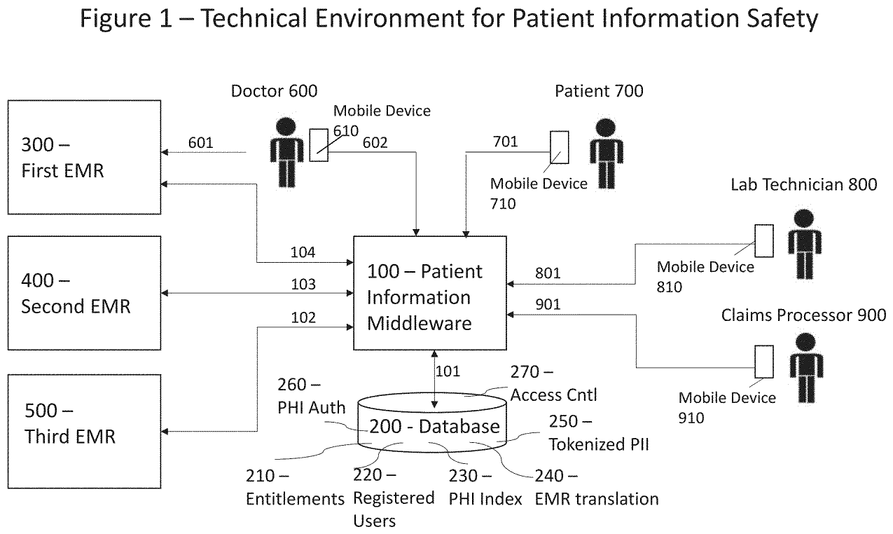

FIG. 1 is an overview of the environment required to support the primary embodiments. A Patient Information Middleware Software (100) is operable to provide need-to-know access to medical records using communication link (101) based on configurations comprised within database (200). Database (200) is further comprised of a User Entitlements table (210), Registered Users table (220), PHI Index table (230), EMR Translation table (240), a Tokenized PII table (250), a PHI Auth table (260), and an EMR Access Control table (270). The User Entitlements table (210) is comprised of a set of rules which define and cross reference medical procedures, medical diagnosis, user types, and user data requirements based on a plurality of medical procedures and diagnosis codes and a plurality of entitlements. Registered Users table (220) is comprised of each known user to the system and may include doctors, nurses, laboratory technicians, patients, claims processors and other participants in the HIPAA business associate eco-system. Each user is assigned a role within the system corresponding to their job type or data ownership. The PHI Index (230) is a cross-reference table that tracks the location and type of medical record information over a variety of separate EMR systems. A given patient may have heart rate and blood pressure data stored on multiple EMR systems and surgical history stored on another EMR system, for example. The PHI Index provides a complete list of each EMR where the patient's data is stored and the associated dates of diagnosis and procedures performed. Data is stored in a hashed and tokenized format. The tokenized PHI values are calculated separately for each record of the PHI Index (230) using the Tokenization Layer (550) of the Patient Information Middleware. The EMR Translation (240), is a database table that defines the APIs, workflows, and message formats related to separate EMR systems. A workflow and message format is associated with each API. The Patient Information Middleware Software (100) can use this database table to determine how to access and transform specific medical records stored on specific EMR systems as reflected in the PHI Index (230).

Tokenized PII (250) is operable to store hashed and encrypted values in place of a patient's personally identifiable information such as the patient's name, date of birth, address or social security number. This hashed and tokenized PII value is used in lieu of actual PII data when requesting medical records; wherein a tokenized patient ID is also stored with each unique row of tokenized PII data. The Patient Information Middleware Software (100) reads the Tokenized PII table to find each unique Tokenized patient ID for a given patient. The tokenized patient ID is the same tokenized patient ID that is stored in the PHI Index table (230) and associated with the provider EMR systems that comprises the patient's official medical records.

A First EMR System (300) is operable to store a variety of structured and un-structured medical records related to a sub-group of patients. A Second EMR System (400) is operable to store a variety of structured and un-structured medical records related to a second sub-group of patients. A Third EMR System (500) is operable to store a variety of structured and un-structured medical records related to a third sub-group of patients. A given patient may have one or more medical records stored within one or more sub groups of patient medical records. Mr. Doe's information, for example, may be spread over all three or more EMR systems; wherein the blood work from past physical exams is stored on EMR System (300), the frozen shoulder procedure history is stored on EMR System (400), and the visit with the Urologist is stored on EMR System (500).

Each EMR system is operable to provide access to medical records based on tokenized requests from the Patient Information Middleware (100) as reflected in Links (104, 103, 102). PHI Auth (260) contains records that pertain to requests for medical records that have been authorized by the Patient Information Middleware (100). A tokenized request is a request for PHI information from a mobile device of a doctor using a tokenized PII value. According to a common definition as disclosed in www.Wikipedi.com, tokenization when used in this manner is the process of substituting a sensitive data element with a non-sensitive equivalent, referred to as a token that has no extrinsic or exploitable meaning or value. The Patient Information Middleware (100) is operable to transform a tokenized request received from a doctor into a patient record number native to the provider EMIR using the patient IDs stored on the Tokenized PII table (250).

Doctor (600) is a registered user within the system. Data is accessed by Doctor (600) using Mobile Device (610) and link (602). Mobile Device (610) may be a smart phone such as an iPhone, Android phone, Blackberry or other similar mobile device or computing device capable of supporting other embodiments. The primary embodiment comprised within the mobile device is the HIPAA Security Application. Other embodiments comprised within the Mobile Device include Bluetooth low energy (“BLE”) Antenna operable to receive a BLE signal, a Secure Element operable to store doctor and patient credentials. The HIPAA Security Application may be deployed as an applet into the Secure Element or it may be deployed as an application comprised within the memory of the mobile device. Doctor (600) may also have direct access to a specific EMR (such as EMR (300)); wherein data is accessed by Doctor (600) using communication link (601) without the need for Mobile Device (610). In this case, EMR 300 is the default provider EMIR of Doctor (600). Patient (700) is a registered user within the Patient Information Middleware Software (100). Data is accessed by Patient (700) using a mobile device and (710) and communication link (701). Lab Technician (800) is a registered user within the Patient Information Middleware Software (100). Data is accessed by Lab Technician (800) using mobile device (810) and communication link (801). Claims Processor (900) is a registered user within the Patient Information Middleware Software (100). Data is accessed by Claims Processor (900) using mobile device (910) and communication link (901). Each registered user may obtain medical records from the system based on their defined role and entitlements related to their job requirements or data ownership. A patient, for example, may be entitled to access any personal medical record irrespective of where that data is stored. Using this system, a given patient such as Mr. Doe may also request a report that shows an aggregated view of his personal health information and the location of each record and any differences between the data stored in different EMR systems.

Each of the EMR Systems (300, 400, 500) and the Patient Information Middleware System (100) consists of a series of components including CPUs, virtual machines, firewalls, load balancers, and storage devices. The storage devices may be dedicated servers with dedicated storage disks or the storage devices may be an attached storage area network (SAN). The servers may be dedicated physical servers such as an HP ProLiant ML350p Gen8-Xeon E5-2670V2 2.5 GHz-32 GB or the servers may be virtualized servers. The server operating system may be one of a Unix, Linux, Windows or another operating system. The database comprised within an EMR system and Database (200) may be one of MySQL®, Oracle®, DB2®, MS SQL® server or another appropriate database to comprise the tables and transactions of the Patient Information Middleware System (100). A web services layer is typically used to enable the graphical user interface or presentation layer for the server application. The presentation layer may be created with one of pHp, html, .net or other appropriate languages and approaches. The web services layer also is operable to handle data sent from end points using application programming interfaces (APIs).

Communication links 101, 102, 103, 104, 601, 602, 701, 801 and 901 may be implemented using any number of common technologies such as BLE, WiFi, HTTP, HTTPS, SSH, FTP, SFTP and other appropriate transport protocols and using proprietary, ISO, restful, xml or other message formats. Messages may be either synchronous or asynchronous messages. Mobile devices 710, 810, and 910 are operable to support the primary embodiments required to access information using software installed on the mobile device in communication with the Patient Information Middleware (100).

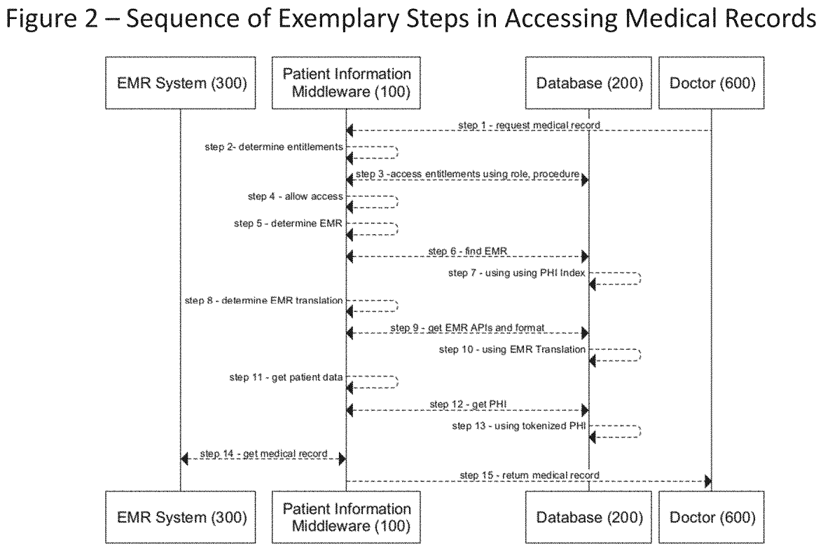

FIG. 2 is a sequence of the exemplary steps involved using the embodiments described in FIG. 1 in granting access to data requested from Doctor (600) of a medical record stored in an EMR System (300) using Patient Information Middleware (100) based on entitlement or ownership as defined in User Entitlements table (210) of Database (200). EMR System (300) is the equivalent of EMR Systems (300) previously described in FIG. 1 and can be any EMR system that contains a subset of patient information. The plurality of components described in FIG. 1 work together to accomplish the following sequence of steps: Step 1—request medical record: is made by Doctor (600) in connection with a medical procedure code assigned to the Doctor. The request for medical records is transmitted from the mobile device (FIG. 1, 610) of the doctor and comprised of the doctor's tokenized credentials, one or more scheduled medical procedures which have been assigned to the doctor, the requested medical records, and the tokenized PII of the patient. The medical record is requested using link (FIG. 1, 602) and is received by an API of the Patient Information Middleware (100). Step 2—determine entitlements: is automatically performed by the Patient Information Middleware (100) on receipt of the request using a workflow comprised within the Patient Information Middleware (100); where the workflow is associated with the API. Step 3—access entitlements using role, procedure: reads Database (200) User Entitlements table (210) using doctor's credentials to determine if Doctor (600) needs the requested medical records in connection with the scheduled medical procedure and in accordance with the role of ‘doctor’. The criteria of role-based access is also determined by the specialty of the doctor. Step 4—allow access: Patient Information Middleware (100) allows access to the requested medical records. Step 5—determine EMR: is operable to locate the specific EMR system where the relevant medical records are stored. Step 6—find EMR: is exemplary of the step to determine the location of the EMR system. Step 7—using PHI Index: illustrates the function of the PHI Index (previously described in FIG. 1) to determine the location of the EMR system with the requested medical record. In Step 7, the Tokenized PII of the patient received in the request message from the doctor is used to read the Tokenized PII table (250) of the Database (200) to find each row that matches the Tokenized PII value of the patient. For each matching row, the Patient Information Middleware (100) reads the value in the column corresponding to the Tokenized Patient ID of the patient and creates in the memory of computer an array of unique Tokenized Patient ID values. This array of unique Patient ID values is then used to read the PHI Index (230) table to find all corresponding rows with matching Tokenized Patient IDs. A row of data is written to the PHI Auth table (260) corresponding to each user and the procedure. Steps 8, 9 and 10 work together. Step 8—determine EMR translation: is a subroutine operable to determine the method to access medical records on the identified EMR System (300). Step 9—get EMR APIs and message format: is requested in connection with the subroutine of Step 8 to read the Database (200). Step 10—using EMR Translation: exemplifies physically reading EMR Translation table (240). The result of Step 10 returns the API number, workflow number and message format number associated with EMR System (300) to the Patient Information Middleware (100) for use in subsequent processing. Steps 11, 12, and 13 work together. Step 11—get patient data: is a subroutine operable to access the required medical records stored on EMR System (300). Step 12—get PHI: is requested by the subroutine of step 11 to read the Database (200), PHI Index table (230) to retrieve the relevant rows of data in accordance with the request and to read the Access Control table (270) to identify the IP address and credentials associated with EMR System (300). Step 13—exemplifies the use of Tokenized PHI data retrieved from the PHI Index table to create a list of records to obtain from the EMR System (300). Step 14—get medical record: exemplifies the access into EMR System (300) using the API, workflow and message provided in Steps 8-10, and the Tokenized PHI, and IP address and credentials provided in Steps 11-13; resulting in the return of the requested medical records. The Tokenized PHI may be de-tokenized in connection with this step wherein the Patient ID is transformed back into its original format associated with the EMR System (300). In an alternate embodiment, the data request of Step 14 is sent to the EMR System (300) in a tokenized format. In this case, the target EMR System (300) is operable to de-tokenize the data upon receipt using an API of the Patient Information Middleware (300). Step 15—return medical record: shows the final step in the process whereby the Doctor (600) received the requested medical records.

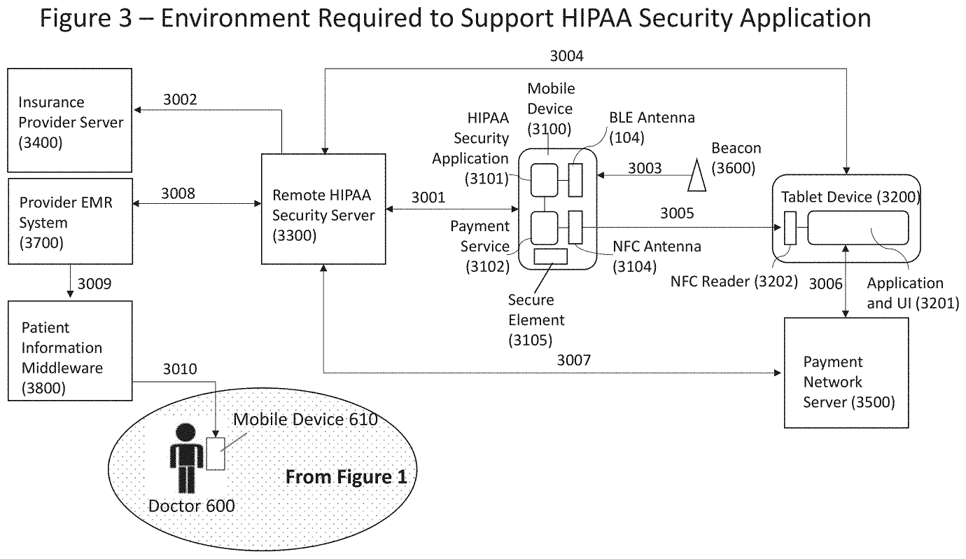

FIG. 3 is an overview of the environment required to support the primary embodiments needed to support the HIPAA Security Application. Mobile Device (3100) may be a smart phone such as an iPhone, Android phone, Blackberry or other similar mobile device capable of supporting other embodiments. The primary embodiment comprised within the mobile device is the HIPAA Security Application (3101). Other embodiments comprised within the Mobile Device (3100) include an installed Payment Service (3102). Payment Service (3102) may be one of ApplePay®, SamsungPay®, AndroidPay® or other commercially available or customized NFC payment services. BLE Antenna (3104) is operable to receive a BLE signal from Beacon Device (3600) as shown in link (3003). Secure Element (3105) is operable to store payment credentials used by Payment Service (3102). HIPAA Security Application (3101) and Payment Service (3102) may be deployed as applets into the Secure Element (3105) or they may be deployed as applications comprised within the memory of the mobile device.

Remote HIPAA Security Server (3300) is operable to communicate with HIPAA Security Application (3101) using communication link (3001). Remote HIPAA Security Server (3300) is operable to communicate with Insurance Provider Server (3400) using communication link (3002). Remote HIPAA Security Server is also operable to communicate with Tablet Device (3200) using communication link (3004) and communicate with Payment Network Server (3500) using communication link (3007). Insurance Provider Server (3400) is operable to confirm insurance coverage on behalf of patients based on message requests received from Remote HIPAA Security Server (3300). Payment Network Server (3500) is operable to receive and process card-not-present payment requests from Remote Server (3300) using link (3007). Payment Network Server (3500) is also operable to receive and process card-present payment requests from the Tablet Device (3200) using communication link (3006). It is therefore a novelty and advantage of the HIPAA security solution to support both card-present or card-not-present payment methods.

Tablet Device (3200) is comprised of NFC Reader (3202) and Application and User Interface (3201). Application and User Interface (3201) is operable to receive messages from Remote HIPAA Security Server (3300) corresponding to patient check-in, insurance verification, HIPAA disclosure verification, card-not-present payment confirmation. Application and User Interface (3201) is also operable to receive NFC payment credentials transmitted from mobile device to NFC Reader (3202) using link (3005). NFC payment credentials are forwarded to Payment Network Server (3500) using communication link (3006). Traditional card-based payments are also supported by the Tablet Device (3200) using available card-acceptance peripheral devices (not shown).

Provider EMR System (3700) is operable to store the medical records of patients using the unique patient ID associated with the EMR system of the current provider and to record an acknowledgement from the patient regarding the patient's approval to release medical records through the Patient Information Middleware (3800); wherein medical records are only released to authorized medical care providers based on the need-to-know and wherein the minimum number of medical records are released in accordance with the patient's current medical condition or required treatment and in accordance with user entitlements. Patient Information Middleware (3800) was previously described in FIG. 1 and is operable to receive a message (3009) from the Provider EMR System (3700) with an update of the patient record to be applied to the PHI Index of the patient receiving care. Message (3009) is comprised of a patient record that also includes an indication of the provider EMR system, the patient ID, the medical procedures, diagnosis, and associated dates. Responsive to the update message, Patient Information Middleware (3800) is operable to parse the medical record into individual components, hash and tokenize the data and update the PHI Index table (230). After the update is complete, the updated PHI Index (230) table is operable to process a request (such as described above) for at least one new medical record corresponding to the Message (3009).

Each of the Remote Server (3300), Payment Network Server (3500), and Insurance Provider Server (3400) and Provider EMR System (3700) consists of a series of components including CPUs, virtual machines, firewalls, load balancers, and storage devices. The storage devices may be dedicated servers with dedicated storage disks or the storage devices may be an attached storage area network (SAN). The servers may be dedicated physical servers such as an HP ProLiant ML350p Gen8-Xeon E5-2670V2 2.5 GHz-32 GB or the servers may be virtualized servers. The server operating system may be one of a Unix, Linux, Windows or another operating system. The database may be one of MySQL®, Oracle®, DB2®, MS SQL® server or another appropriate database to comprise the tables and transactions of the HIPAA Wallet. A web services layer is typically used to enable the graphical user interface or presentation layer for the server application. The presentation layer may be created with one of pHp, html, .net or other appropriate languages and approaches. The web services layer also is operable to handle data sent from end points using application programming interfaces (APIs).

Communication links 3001, 3002, 3003, 3004, 3005, 3006 and 3007 may be implemented using any number of common technologies such as BLE, WiFi, HTTP, HTTPS, SSH, FTP, SFTP and other appropriate transport protocols and using proprietary, ISO, restful, xml or other message formats. Messages may be either synchronous or asynchronous messages.

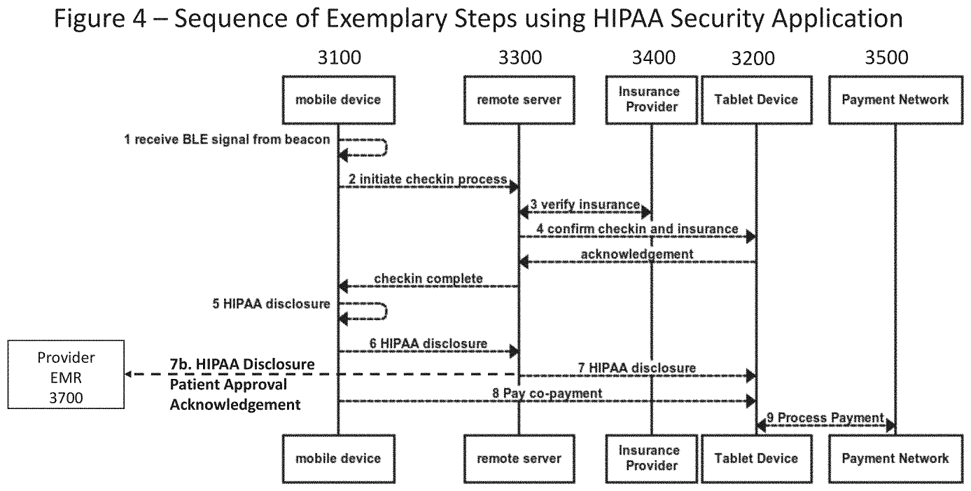

FIG. 4 is a sequence of the exemplary steps involved when using the embodiments described in FIG. 3 in connection with a typical use case. Step 1—Mobile Device (3100) is comprised of a HIPAA Security Application and operable to receive a BLE signal from a beacon device (3103), BLE signal indicating the identity and location of the medical care provider and causing the HIPAA Security Application to initiate a check-in sequence. Step 2—HIPAA Security Application comprised within Mobile Device (3100) sends communication to Remote HIPAA Security Server (3300), communication message comprised of tokenized patient PII information and one or more of the identity and location of the medical care provider. Remote HIPAA Security Server (3300) reconciles tokenized patient PII information to the actual patient unique identity and the previously stored insurance policy information for patient. Tokenized patient PII information is also used to determine the Patient ID associated with the Provider EMR system. A database table such as the Tokenized PII (250) database table comprised within the Patient Information Middleware is used for this translation. In Step 3—Remote HIPAA Security Server (3300) sends communication message to Insurance Provider (3400) with insurance policy information (e.g. the insurance eligibility check). Step 4—after having received positive confirmation from Insurance Provider (4220), Remote HIPAA Security Server (3300) communicates one or both of patient check-in and insurance confirmation to Tablet Device (3200) resulting in an acknowledgement message back from Tablet Device (3200) to Remote HIPAA Security Server (3300). Acknowledgement message traverses back to HIPPA Security Application comprised within the Mobile Device (3100). Step 5—HIPAA Security Application comprised within Mobile Device (3100) is operable to initiate a HIPAA disclosure sequence wherein the patient approves the release of applicable medical records related to the approved procedures. However, only those medical records related to the approved procedures are authorized for release. It is thus an advantage of the HIPAA Security Application to release the minimum medical records required. Step 6—HIPAA disclosure message is sent to the Remote HIPAA Security Server (3300). During Step 6, Provider EMR System (3700) is updated to reflect the patient's approval to release medical records (using link 3008 described in FIG. 3). Step 7—HIPAA disclosure message is sent to the Tablet Device (3200). Step 7b HIPAA disclosure message sent to the EMR system (3700) of the provider to record an acknowledgement from the patient regarding the patient's approval to release medical records to authorized medical care providers based on the need to know wherein the minimum number of medical records are released in accordance with the patient's current medical condition or treatment. Step 8—HIPAA Security Application comprised within the Mobile Device (3100) in communication with installed NFC payment service, initiates a payment transaction with Tablet Device (3200) to pay the co-payment amount due to the medical care provider. Step 9—Tablet Device (3200) completes payment transaction with Payment Network (3500).

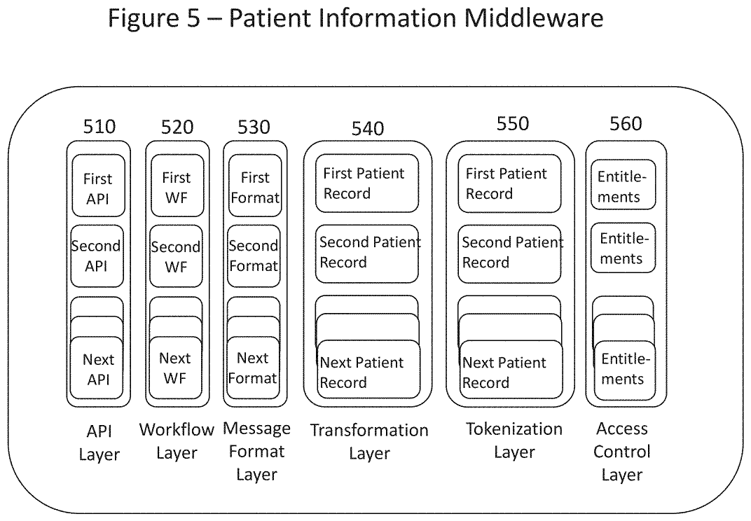

FIG. 5 provides additional details about the Patient Information Middleware (100) which is comprised of six primary software layers, each layer performing a separate function of the middleware. API Layer (510) is comprised of compiled software designed and operable to access medical records stored in designated provider (e.g ‘target’) EMR systems as determined by the Patient Information Middleware (100) using the APIs comprised within EMR Translation table (240). Workflow Layer (520) is comprised of compiled software designed and operable with the specific steps required to access medical records stored in a designated provider EMR system as determined by the Patient Information Middleware (100) using the workflows comprised within EMR Translation table (240). Message Format Layer (530) is comprised of compiled software designed and operable to understand the format of medical records stored in provider's target EMR systems using the formats derived from the EMR Translation table (240). Transformation Layer (540) is comprised of compiled software designed and operable to transform received medical records from a provider's target EMR message format into a storage format of the Patient Information Middleware (100); wherein records are transformed in such a way as to separate or parse each received medical record into individual index records related to separate procedures and separate diagnosis including the dates associated with the medical procedures and diagnosis. It is thus an advantage of the system to be able to provide the minimum viable and most recent medical record information from the transformed medical index records. Tokenization Layer (550) is comprised of compiled software designed and operable to tokenize the transformed medical records for storage into the database tables of the Patient Information Middleware (100). Access Control Layer (560) is operable to determine if the medical records and medical history requested are requested in accordance with the data comprised within the User Entitlements table (210) and is operable to update the PHI Auth table (260).

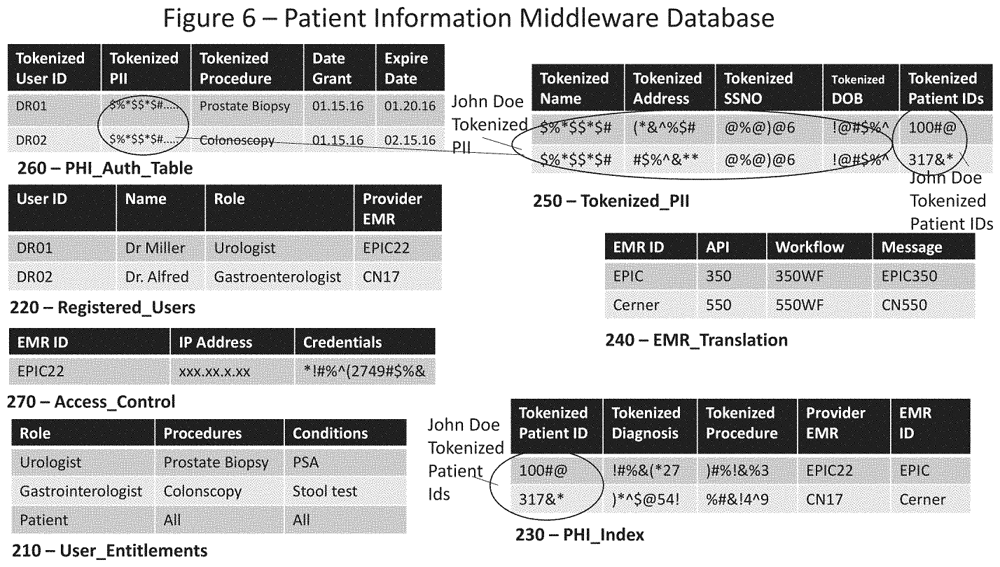

FIG. 6 provides additional details about the tables comprised within FIG. 1, Database (200). The User Entitlements table (210) is comprised of a set of rules which define and cross reference medical procedures, medical diagnosis, user types, and user data requirements based on a plurality of medical procedures and diagnosis codes and a plurality of entitlements. Specific columns in the User Entitlements table can include but are not limited to: role, procedures and conditions. For example, a ‘urologist’ role user may have access to medical records related to a prostate biopsy and conditions related to a patient's PSA test results. In contrast, a ‘gastroenterologist’ role user may have access to medical records related to a colonoscopy and conditions related to a stool test. Finally, a ‘patient’ role may have access to all of their personal medical procedure and conditions based on ownership.

The Registered Users table (220) is comprised of each known user to the system and may include doctors, nurses, laboratory technicians, patients, claims processors and other participants in the HIPAA business associate (BAA) eco-system. Each user is assigned a role within the system corresponding to their job type or data ownership and as defined in the User Entitlements table (210). Columns can include but are not limited to: User ID, Name, Role, and Provider EMR; wherein the provider EMR system is the EMR system tied directly to the doctor's primary practice and wherein the doctor may have direct access to medical records without using the Patient Information Middleware (100).

The PHI Index (230) is a cross-reference table that tracks the location and type of medical record information over a variety of separate EMR systems. A given patient may have heart rate and blood pressure data stored on multiple EMR systems and surgical history stored on another EMR system, for example. The PHI Index provides a complete list of each EMR where the patient's data is stored and the associated dates of diagnosis and procedures performed. Data is stored in a hashed and tokenized format for speed and security using the Tokenization Layer (540) of the Patient Information Middleware. As previously defined, tokenization is the process of substituting a sensitive data element with a non-sensitive equivalent, referred to as a token that has no extrinsic or exploitable meaning or value. A hash value as defined by www.webopedia.com, is a number generated from a string of text. The hash is substantially smaller than the text itself, and is generated by a formula in such a way that it is extremely unlikely that some other text will produce the same hash value. The hashed and tokenized PHI values are calculated separately for each record in the PHI Index (230) wherein the individual medical procedure codes are hashed together with the dates of service and then encrypted as a token and wherein the individual diagnosis codes are hashed together with the date of diagnosis and then encrypted as a token. Tokenization is performed using the Tokenization Layer (550) of the Patient Information Middleware. Columns can include but are not limited to: Tokenized Patient ID, Tokenized Diagnosis, Tokenized Procedure, Provider EMR, and EMR ID. The Tokenized Patient ID is the patient ID related to a specific target EMR system where a medical record exists. An EMR patient ID is tokenized by computing a mathematical hash value that corresponds to the actual patient ID and then encrypting this hashed value. A given patient such as Mr. Doe may have multiple tokenized patient IDs. As shown in FIG. 6, Mr. Doe has a first tokenized patient ID of ‘100#@’ which corresponds to a first ‘EPIC22’ EMR system. Mr. Doe also has a second tokenized patient ID of ‘317&*’ which corresponds to a second ‘CN17’ system. A tokenized diagnosis code of ‘!#%&(*27’ is related to Mr. Doe's medical condition reflected in the patient records stored on the first EMR. A tokenized procedure code of ‘)#%!&%3’ is related to a medical procedure documented on the first EMR. The tokenized diagnosis and tokenized procedure codes include the date of diagnosis or service reflected in the first EMR system. It is therefore an advantage of the PHI Index to provide the ability to quickly find the most recent medical history of a patient using the stored date. The Provider EMR column contains the code associated with the target EMR system that contains the actual patient record. In the example shown in FIG. 6, Mr. Doe had patient records on both the ‘EPIC22’ and ‘CN17’ EMR systems. Finally, the EMR ID column contains the ID associated with the API, workflow, and message format associated with the target EMR. The EMR ID is used by the Patient Information Middleware to access the API, workflow, and message format from the EMR Translation table (240).

The EMR Translation (240), is a database table that defines the APIs, workflows, and message formats related to separate EMR systems. A workflow and message format is associated with each API. The Patient Information Middleware Software (100) can use this database table to access and then transform specific medical records stored on specific EMR systems as reflected in the PHI Index (230). Transformation is performed using the Transformation Layer (540) of the Patient Information Middleware. Columns can include but are not limited to the EMR No., the API No, the Workflow No., and the Message Format No. In the example shown, the ‘EPIC’ EMR system is accessed using the API No. ‘350’, Workflow No. ‘350WF’, and message format ‘EPIC350’.

The Tokenized PII table (250) is operable to store hashed and encrypted values in place of a patient's personally identifiable information such as the patient's name and address, date of birth, or social security number. This tokenized PII value is used in lieu of actual PII data by doctors when requesting medical records using mobile devices. A tokenized Patient ID is also stored as a separate column with each unique row of Tokenized PII data. The patient ID is tokenized by computing a mathematical hash value that corresponds to the patient ID and then encrypting this hashed value. The Patient Information Middleware Software (100) reads the Tokenized PII table to find each unique tokenized patient ID for a given patient. The tokenized patient ID is the same tokenized patient ID that is stored in the PHI Index table (230) and associated with the provider EMR systems that comprises the patient's actual medical records. A given patient such as Mr. Doe may have multiple unique tokenized patient IDs; wherein each unique tokenized patient ID corresponds to a different target EMR system. The columns of the Tokenized PII table (250) include but are not limited to: Tokenized Name, Tokenized Address, Tokenized Date of Birth, Tokenized SSNO, and Tokenized Patient ID. Taken together, the Tokenized Name+Tokenized Address+Tokenized SSNO+Tokenized Date of Birth constitute the Tokenized PII of a specific patient. Data on the Tokenized PII table may be accessed using a single column of tokenized PII data or any combination of columns. In the example shown in FIG. 6, Mr. Doe's tokenized PII is represented as ‘$%*$$*$##$%\hat{ }&**@%@)@6!@#$%\hat{ }’. This Tokenized PII value is associated with two unique tokenized patient IDs for Mr. Doe represented as ‘100#@’ and ‘317&*’. These unique tokenized patient IDs are the same tokenized patient IDs represented in the PHI Index table (230) for Mr. Doe.

PHI Auth (260) contains records that pertain to requests for medical records that have been authorized by the Patient Information Middleware (100) Access Control Layer (560). A tokenized request is a request for PHI information of a patient using a tokenized PII value obtained from the mobile device of an authorized user such as a doctor or BAA. The Patient Information Middleware is operable to transform a tokenized request received from an authorized user comprised of a PII value for a given patient into a patient record number native to the provider EMR using the tokenized patient IDs stored on the Tokenized PII table (250). The columns in the PHI Auth table (260) include but are not limited to: Tokenized User ID, Tokenized PII, Tokenized Procedure, Date Grant, and Expire Date. In the example shown in FIG. 6, ‘DR01’ is granted access to PHI information of Mr. Doe for a Prostate Biopsy that has been scheduled for the patient and assigned to the doctor corresponding to Tokenized User ID ‘DR01’. The Tokenized PII of Mr. Doe is represented as ‘$%*$$*$##$%\hat{ }&**@%@)@6!@#$%\hat{ }’. As previously discussed, this Tokenized PII value can be used to find the unique Tokenized Patient IDs related to Mr. Doe on the Tokenized PII table (250) and then again to retrieve the target EMR systems from PHI Index table (230). In the same example, the User ‘DR01’ was granted access to the medical records needed to properly perform a Prostate Biopsy on the Grant Date of ‘011516’. This access privilege is valid until the Expire Date of ‘01.20.16’.

The Access Control table (270) is used by the Patient Information Middleware (100) to gain access to the target EMR system determined from the PHI Index table (230). The columns of this table include but are not limited to: EMR ID, IP Address, and Credentials; wherein the credentials are stored in an encrypted format.

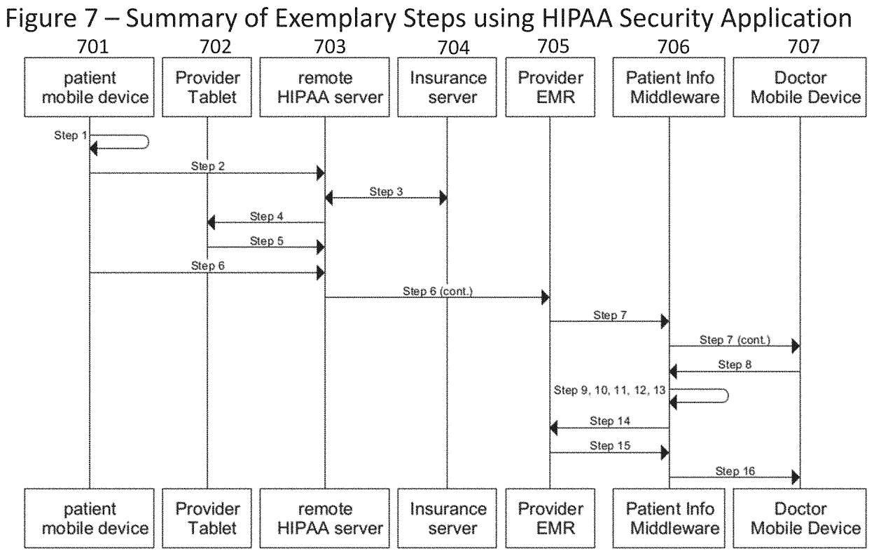

We have therefore described an apparatus, method, and system for patient information safety comprised of a plurality of electronic medical record systems, a plurality of patients, a plurality of doctors, a plurality of lab technicians, a plurality of claims processors, and further comprised of a patient information middleware comprised of a plurality of CPUs, virtual machines, firewalls, load balancers, and storage devices; and further comprised of a database which is formatted to store user entitlements, registered users, a PHI index, EMR translations, and tokenized PII information; and further comprised of a plurality of patient mobile devices, each patient device operable to receive BLE signals and further equipped with a secure element operable to store payment credentials and further comprised of a HIPAA security application and a payment service application; environment further comprised of a remote server operable to communicate with the HIPAA security application using a communication link; environment further comprised of an insurance provider server operable to confirm insurance coverage; environment further comprised of a tablet device comprised of NFC reader, application and user interface wherein the application and user interface are operable to receive credentials transmitted from the patient mobile device to NFC reader; and environment further comprised of a payment network server operable to receive and process payment requests from a plurality of tablet devices and wherein the components of the system are operable together for performing the following sequence of steps as shown in FIG. 7.

In Step 1, a mobile device (701) of a patient comprised of a HIPAA security application receiving a BLE signal from a beacon transmitting device, BLE signal indicating the identity and location of the medical care provider and causing the HIPAA security application to initiate a check-in sequence.

In Step 2, responsive to the check-in sequence requirements, the HIPAA security application comprised within mobile device sending a communication message to the remote HIPAA security server (703), the communication message including tokenized patient PII information and one or more of the identity and location of the medical care provider; tokenized patient PII information derived from a secure element or secure memory of the mobile device of the patient.

In Step 3, the remote HIPAA security server responsive to check-in message received from the mobile device, reconciles tokenized patient PII information to stored tokenized patient PII information using the patients PII to determine the unique tokenized patient ID associated with the EMR system of the current provider and determines insurance coverage for patient and wherein the remote HIPAA security server sends a communication message to insurance provider server (704) with insurance policy information of the patient.

In Step 4, after having received confirmation from the insurance provider server, the remote HIPAA security server communicates one or both of patient check-in and insurance confirmation messages to tablet device (702) resulting in an acknowledgement message sent back from the tablet device to the remote HIPAA server (703).

In Step 5, an acknowledgement message traverses back to the HIPPA security application comprised within the mobile device (701) of the patient.

In Step 6, wherein the HIPAA security application comprised within mobile device (701) is operable to initiate a HIPAA disclosure sequence whereby the patient approves the release of applicable medical records related to the approved procedures and wherein the EMR system (705) of the provider is updated to reflect the patient's approval to release the medical records.

In Step 7, wherein responsive to the patient's need for medical care, the EMR system (705) transmitting the patient's tokenized PHI and required procedures to the patient information middleware (706); tokenized PHI index records stored in the database of the patient information middleware and associated with the patient; patent information middleware determining patient's tokenized PII and transmitting patient's tokenized PII and required procedures to a mobile device (707) of a doctor assigned to the patient.

In Step 8, the patient information middleware (706) receiving a tokenized request for a medical record made by the assigned doctor in connection with the required medical procedure; the tokenized request including the doctor's tokenized ID, the tokenized PII of the patient, at least one medical procedure code; the medical record request received by an API of the patient information middleware. Step 8 handled using the API Layer (510).

In Step 9, responsive to the request, the patient information middleware (706) determining entitlements of the doctor using the doctor's role and the scheduled medical procedure code by reading the user entitlements comprised within the database to determine if the doctor needs the requested medical record in connection with the medical procedure associated with the request and in accordance with the role of a doctor; wherein the role of doctor is further refined by the specialty of the doctor. Step 9 performed by the Access Control Layer (560) and using the User Entitlements table (120).

In Step 10, the patient information middleware (706) allowing access to the requested medical record in accordance with the entitlements determined for the specialty of the doctor and the medical procedure code; access granted by updating the database of the patient information middleware to create at least one row of data in the PHI Auth table (260) comprised of the doctor's tokenized ID, the patient's tokenized PII; each row of the database also comprised of a date and time stamp indicating the grant date and an expiration date associated with the access rights. Step 10 performed by the Access Control Layer (560) and using the User Entitlements table (120).

In Step 11, the patient information middleware (706) using the tokenized patient IDs comprised within the Tokenized PII table (250) to find matching rows stored in the PHI Index table (230); locating the specific target EMR system where the relevant medical record is stored by using the provider EMR code comprised within the PHI Index table of the database as a cross reference to the EMR Translation table (240); PHI Index table (230) of the database comprised of a plurality of tokenized patient IDs, an indicator for each provider EMR system where associated medical records exist for the patient associated with the tokenized patient ID, and the most recent dates associated with each medical procedure and medical diagnosis conducted on behalf of the patient.

In Step 12, the patient information middleware (706) determining the method to access medical records on the identified EMR system using the EMR ID derived from the PHI Index table (230) to find the corresponding EMR access requirements on the EMR Translation table (240); EMR access method comprised of API definitions, related workflows for each API, message formats, and security procedures; wherein security procedures include at least the IP address and login credentials of the target EMR; EMR access requirements determined from the Access Control table (270) using the EMR provider number from the PHI Index (230); and wherein the firewall associated with the target Provider EMR has been configured to permit this request from the patient information middleware.

In Step 13, the patient information middleware (706) transforming the tokenized patent ID from the PHI Index table into a patient record ID format which can be used with the target EMR System. Step 13 completed by the Transformation Layer (540).

In Step 14, the patient information middleware requesting the medical record from the target Provider EMR System (705) using the identified APIs, record formats and patient record ID; wherein the patient information middleware accesses the target Provider EMR system (705) using the security procedures, APIs and message formats derived from the database; wherein the data request is transformed in accordance with the required record format of the target Provider EMR (705) system. Step 14 completed by the API Layer (510), Workflow Layer (520) and Message Format Layer (530).

In Step 15, the patient information middleware system (706) receiving the medical record from the target Provider EMR system (705) in accordance with the prior approval of a patient;

In Step 16, the patient information middleware system (706) returning the minimum viable medical records to the mobile device (707) of the doctor in accordance with the request and prior authorization of the patient.

We claim:

1. A system for patient information safety comprising a patient information middleware software module operable to provide controlled access to medical records based on configurations stored within a database; the database further comprising a user entitlements table, a registered users table, a patient health information index table, an electronic medical record translation table, a tokenized personally identifiable information table, and a patient health information authorization table;
the user entitlements table comprising a set of rules which define and cross reference medical procedures, medical diagnoses, user types, and user data requirements based on a plurality of medical procedures and diagnosis codes and a plurality of entitlements;
the registered users table comprising users of the system including a plurality of doctors, nurses, laboratory technicians, patients, and claims processors, wherein each user is assigned a role within the system corresponding to a job type or a data ownership;
the patient health information index table deployed as a cross-reference table that tracks a location and a type of medical record information over a plurality of electronic medical information systems; wherein the patient health information index table provides a list of electronic medical record systems of the plurality of electronic medical information systems where data associated with a patient is stored together with associated dates of diagnosis and procedures performed; wherein the patient's data is stored as a hashed and tokenized value; wherein the hashed and tokenized value is calculated separately for each record of the patient's data using a tokenization layer of the patient information middleware software module;
wherein the electronic medical record translation table defines an application programming interface and an associated workflow and message format related to each of the plurality of electronic medical record systems; wherein the patient information middleware software module uses the electronic medical record translation table to access and transform a first medical record of the patient stored on a first electronic medical information system as reflected in the patient health information index table;
the system causing the following steps to occur when a processor executes computer-readable instructions of the patient information middleware software module:
receiving, at an application programming interface (API) software layer module of the patient information middleware software module, a tokenized request for the first medical record received from a doctor's computing device, the tokenized request including a medical procedure code and tokenized patient personally identifying information data;
responsive to receiving the tokenized request, the patient information middleware software module accessing the user entitlements table and determining entitlements of the doctor using the medical procedure code and a role for the doctor;
the patient information middleware software module allowing access to the first medical record based on the entitlements of the doctor;
the patient information middleware software module using the tokenized request to locate matching tokenized patient personally identifiable information data in the tokenized personally identifiable information table and create in a memory a tokenized patient identifier to read the patient health information index table to locate the first medical record stored on the first electronic medical information system;
the patient information middleware software module using the API software layer module, a workflow software layer module, and a message format software layer module to access and retrieve the first medical record;
the patient information middleware software module using the electronic medical record translation table to translate the first medical record;
the patient information middleware software module transforming the tokenized patient personally identifiable information data in the tokenized request into a patient record ID that is used with the first electronic medical information system; and
the patient information middleware software module using the transformed patient record ID to provide the translated medical record to the doctor's computing device.

2. The system of claim 1, wherein the system further causes the following steps:
transmitting a bluetooth low energy signal from a beacon transmitting device located at a medical care provider to a patient mobile computing device, the bluetooth low energy signal indicating an identity and a location of the medical care provider; and
responsive to receiving the bluetooth low energy signal at the patient mobile computing device, a HIPAA security application stored on the patient mobile computing device initiating a check-in sequence that comprises the HIPAA security application transmitting the tokenized patient personally identifiable information data and one or more of the identity and the location of the medical care provider to a HIPAA security server.

3. The system of claim 2, wherein the system further causes the following steps:
responsive to receiving the tokenized patient personally identifiable information data at the HIPAA security server from the HIPAA security application stored on the patient mobile computing device, the HIPAA security server reconciling the tokenized personally identifiable information data into patient personally identifiable information data for identifying insurance policy information associated with the patient and stored at the HIPAA security server; and
transmitting a request from the HIPAA security server to an insurance provider server to confirm insurance coverage for the patient.

4. The system of claim 3, wherein the system further causes the following steps:
transmitting, from the HIPAA security server to a medical care provider computing device one or more of an insurance coverage confirmation message and a check-in sequence confirmation message.

5. The system of claim 2, wherein the HIPAA security application stored on the patient's mobile computing device receives from the HIPAA security server an acknowledgement message.

6. The system of claim 2, wherein the system further causes the following steps:
the HIPAA security application initiating a HIPAA disclosure sequence whereby the patient approves a release of a medical record related to the medical procedure code; and
an electronic medical information system of the medical care provider is updated to reflect an approval to release the medical record.

7. The system of claim 6, wherein the system further causes the following step:
the electronic medical information system transmitting a tokenized patient protected health information (“PHI”) to the patient information middleware software module.

8. The system of claim 7, wherein the system further causes the following steps:
the patient information middleware software module determining the tokenized patient personally identifiable information data and transmitting the tokenized patient personally identifiable information data to the doctor's computing device.

9. The system of claim 1, wherein the step of allowing access to the medical record is controlled by an access control software layer module of the patient information middleware software module, and wherein upon allowing access to the medical record, a patient health information authorization table is updated with a new record comprising a tokenized doctor identifier, the tokenized patient personally identifiable information data, a date that access was allowed, and an expiration date for access to the medical record.

10. The system of claim 4, wherein the system further causes the following step:
the HIPAA security application initiating a payment transaction with a payment service installed on the patient mobile computing device, the payment service communicating with the medical care provider computing device.

11. The system of claim 10, wherein the medical care provider computing device completes the payment transaction with a payment network server.

12. The system of claim 2, wherein the patient information middleware software module operates on a computer server.

13. The system of claim 2, wherein the patient information middleware software module communicates with one or more of a mobile computing device of a claims processor and a mobile computing device of a lab technician.

14. The system of claim 1, wherein the API software layer module uses the application programming interface stored in the electronic medical record translation table to access the first medical record stored on the first electronic medical information system.
